(19) United States
(12) Patent Application Publication
(10) Pub. No.: US 2002/0091283 A1
(43) Pub. Date: Jul. 11, 2002

(54) Production process for hydroxylalkyl (meth)acrylate

(76) Inventors: Hajime Matsumoto, Himeji-shi (JP); Tetsuya Kajihara, Himeji-shi (JP); Yukihiro Yoneda, Himeji-shi (JP)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/003,044

(22) Filed: Dec. 6, 2001

(30) Foreign Application Priority Data
Jan. 10, 2001 (JP) 2001-002821

(51) Int. Cl. B01D 3/34; C01D 1/00; B01D 3/00; C07B 31/00; B01D 59/00; C07C 67/26

(52) U.S. Cl. 560/209; 203/8

(57) ABSTRACT

The present invention provides a novel production process for a hydroxyalkyl (meth)acrylate in which, when the hydroxyalkyl (meth)acrylate is produced by carrying out a reaction between (meth)acrylic acid and an alkylene oxide, the unreacted (meth)acrylic acid can be effectively recovered and thereafter recycled as a raw reaction material. The production process for a hydroxyalkyl (meth)acrylate comprises the step of carrying out a reaction between (meth)acrylic acid and an alkylene oxide in order to produce the hydroxyalkyl (meth)acrylate, and further comprises the steps of: recovering the unreacted (meth)acrylic acid by distillation of the resultant reaction liquid; and thereafter recycling the recovered unreacted (meth)acrylic acid as a raw material for the reaction.

### BACKGROUND OF THE INVENTION

A. Technical Field

The present invention relates to a production process for a hydroxyalkyl (meth)acrylate, which comprises the step of carrying out a reaction between (meth)acrylic acid and an alkylene oxide.

B. Background Art

When a bydroxyalkyl (meth)acrylate is produced by carrying out a reaction between (meth)acrylic acid and an alkylene oxide, there is hitherto a problem forming an alkylene glycol di(meth)acrylate (hereinafter, referred as diester) or a dialkylene glycol mono(meth)acrylate as a by-product.

Accordingly, the examination is carried out so as to decrease these by-products. As a result, it is reported that the formation of the above by-products is suppressed by controlling the conversion of the (meth)acrylic acid in the range of less than 100% (JP-B-14087/1972, JP-A-26810/1976, and JP-B-30822/1993).

On the other hand, it is also known that the conversion of the (meth)acrylic acid is required as close to 100% as possible in order to suppress the contamination of the (meth)acrylic acid to a product, wherein it is difficult to purify and separate the (meth)acrylic acid (JP-A-330320/1998).

When the contamination of the diester or the dialkylene glycol mono(meth)acrylate is permitted in a certain extent, it is unnecessary to lower the conversion of the (meth)acrylic acid too much, and the amount of the unreacted (meth)acrylic acid is little in the reaction liquid. Therefore, if the contamination is in the range that can be permitted even in case of causing the contamination to the product, there is especially no problem.

On the other hand, when the by-production of the diester or the dialkylene glycol mono(meth)acrylate is sufficiently tried to suppress, it is necessary to suppress the conversion of the (meth)acrylic acid sufficiently according to the above method. In this case, the unreacted (meth)acrylic acid remains in the reaction liquid in a comparatively large amount because the conversion of the (meth)acrylic acid is greatly suppressed. Therefore, in consideration of productivity and economy, it is necessary to separate and recover the unreacted (meth)acrylate, and it is more favorably if it can be recycled as a raw reaction material. However, the (meth)acrylic acid has a strong affinity for the hydroxyalkyl (meth)acrylate and a small relative volatility, and further, is easily polymerizable alone or in a mixed solution. Therefore, the technology concerning the separation, recovery and recycling of the unreacted (meth)acrylic acid, was not completed in the past.

Incidentally, the unreacted alkylene oxide also remains in the reaction liquid. However, as to the technology concerning the separation and recovery of this unreacted alkylene oxide, a method, which involves separating by stripping and recovering by absorbing solvents, is disclosed (JP-A-330320/1998, JP-A-240853/1999, Japanese Patent Application No. 2000-22692, and Japanese Patent Application No. 2000-32340).

### SUMMARY OF THE INVENTION

A. Object of the Invention

Accordingly, an object of the present invention is to provide a novel production process for a hydroxyalkyl (meth)acrylate in which, when the hydroxyalkyl (meth)acrylate is produced by carrying out a reaction between (meth)acrylic acid and an alkylene oxide, the unreacted (meth)acrylic acid can be effectively recovered and thereafter recycled as a raw reaction material.

B. Disclosure of the Invention

The present inventors diligently studied to solve the above-mentioned problems. As a result, they found that: a novel production process for a hydroxyalkyl (meth)acrylate, which involves recovering the unreacted (meth)acrylic acid and recycling it, can be established by distillation of the reaction liquid resultant from the reaction between the (meth)acrylic acid and the alkylene oxide.

That is to say, a production process for a hydroxyalkyl (meth)acrylate, according to the present invention, comprises the step of carrying out a reaction between (meth)acrylic acid and an alkylene oxide in order to produce the hydroxyalkyl (meth)acrylate,

with the production process further comprising the steps of: recovering the unreacted (meth)acrylic acid by distillation of the resultant reaction liquid; and thereafter recycling the recovered unreacted (meth)acrylic acid as a raw material for the reaction.

These and other objects and the advantages of the present invention will be more fully apparent from the following detailed disclosure.

### DETAILED DESCRIPTION OF THE INVENTION

First of all, the addition reaction between the (meth)acrylic acid and the alkylene oxide in the production process according to the present invention is explained.

As to the amount of the raw materials as charged in the reaction between the (meth)acrylic acid and the alkylene oxide, the amount of the alkylene oxide is favorably not less than 0.5 mol, more favorably in the range of 0.8 to 5.0 mols, still more favorably 0.9 to 3.0 mols, yet still more favorably 1.0 to 2.0 mols, per 1 mol of the (meth)acrylic acid. In the case where the amount of the alkylene oxide as charged is less than 0.5 mol, there are disadvantages in that the reaction ratio is lowered and by-products are increased. In addition, in the case where the amount of the alkylene oxide as charged is too much, especially more than 5 mols, there are disadvantages in economy.

The (meth)acrylic acid usable in the present invention means acrylic acid and methacrylic acid.

The alkylene oxide usable in the present invention has favorably 2 to 6 carbon atoms, more favorably 2 to 4 carbon atoms. Examples thereof include ethylene oxide, propylene oxide, and butylene oxide. Ethylene oxide or propylene oxide is favorable, and ethylene oxide is particularly favorable.

In the present invention, the reaction between the (meth)acrylic acid and the alkylene oxide can be carried out according to methods generally used for this kind of reaction, and is generally carried out in the presence of a catalyst.

For example, when the reaction is carried out in a batch manner, it is carried out by introducing the liquid alkylene oxide into the (meth)acrylic acid. Then, the alkylene oxide may be added to the (meth)acrylic acid in a lump, continuously, or intermittently. Then, when it is added continuously or intermittently, as is often the case with this kind of reaction, the reaction is continued still after the addition of the alkylene oxide, in other words, the aging is carried out, and thereby the reaction can be completed. In addition, it is not always necessary to initially add the (meth)acrylic acid at one time, and it can be divided to some portions and then added.

In addition, when the reaction is carried out in a continuous manner, it is carried out by continuously adding the (meth)acrylic acid and the liquid alkylene oxide into a reactor such as a tubular or tank reactor, and by continuously extracting the resultant reaction liquid from the reactor. In this case, the catalyst may continuously be supplied together with the raw materials and then continuously be extracted together with the resultant reaction liquid. In case of the reactor such as a tubular reactor, a solid catalyst may be used in a state of filling the reactor, what is called, in a fixed bed manner. In addition, in case of the tank reactor, a solid catalyst may be used in a state of fluidizing together with the reaction liquid in the reactor, what is called, in a fluidized bed manner. In case of these continuous reactions, the reaction liquid may be circulated partially.

As to the addition of the raw (meth)acrylic acid and the raw alkylene oxide to the reactor, they may be added from separate addition lines respectively. They are beforehand blended in a pipe, a line mixer, or a mixing tank before they are added to the reactor, and thereafter they may be added. In addition, when the liquid obtained from the reactor outlet is circulated to the inlet of the reactor, this liquid may be added to the reactor after blending them with the raw (meth)acrylic acid and the raw alkylene oxide. Furthermore, when the unreacted (meth)acrylic acid or the unreacted alkylene oxide is recovered and recycled like the present invention, these liquids may be added to the reactor after blending them with the raw (meth)acrylic acid and the raw alkylene oxide. However, when the (meth)acrylic acid and the alkylene oxide are added from separate addition lines, the molar ratio of the (meth)acrylic acid in the reaction liquid is excess in the neighborhood where the (meth)acrylic acid is added. Therefore, the respective raw materials are beforehand blended in such as a pipe before they are added to the reactor, and then they may be added thereto.

The reaction temperature is usually favorably in the range of 40 to 130° C., more favorably 50 to 100° C. In the case where the reaction temperature is lower than 40° C., the reaction proceeds very slowly and it is apart from a practical level. On the other hand, in the case where the reaction temperature is higher than 130° C., there are disadvantages in that the by-products are increased and the polymerization of the (meth)acrylic acid as a raw material or the hydroxyalkyl (meth)acrylate as a product is caused.

In addition, the reaction may be carried out in a solvent for the purpose of mildly carrying out the reaction. As to the solvent, the following conventional solvents can be used: toluene, xylene, heptane, and octane. The pressure in the reaction system depends upon the kinds of raw materials or the mixing ratio, but the reaction is generally carried out under compressed pressure.

In addition, when the reaction is carried out, conventional polymerization inhibitors can be used. Examples thereof include: phenol compounds, such as hydroquinone, methylhydroquinone, tert-butylhydroquinone, 2,6-di-tert-butylhydroquinone, 2,5-di-tert-butylhydroquinone, 2,4-dimethyl-6-tert-butylphenol, and hydroquinone monomethyl ether; paraphenylenediamines, such as N-isopropyl-N′-phenyl-para-phenylenediamine, N-(1,3-dimethylbutyl)-N′-phenyl-para-phenylenediamine, N-(1-methylheptyl)-N′-phenyl-para-phenylenediamine, N,N′-diphenyl-para-phenylenediamine, and N,N′-di-2-naphthyl-para-phenylenediamine; amine compounds such as thiodiphenylamine and phenothiazine; copper dialkyldithiocarbamates, such as copper dibutyldithiocarbamate, copper diethyldithiocarbamate, and copper dimethyldithiocarbamate; nitroso compounds, such as nitroso diphenylamine, isoamyl nitrite, N-nitroso-cyclohexylhydroxylamine, N-nitroso-N-phenyl-N-hydroxylamine, and their salts; and N-oxyl compounds, such as 2,2,4,4-tetramethylazetidine-1-oxyl, 2,2-dimethyl-4,4-dipropylazetidine-1-oxyl, 2,2,5,5-tetramethylpyrrolidine-1-oxyl, 2,2,5,5-tetramethyl-3-oxopyrrolidine-1-oxyl, 2,2,6,6-tetramethylpiperidine-1-oxyl, 4-hydroxy-2,2,6,6-tetramethylpiperidine-1-oxyl, 6-aza-7,7-dimethyl-spiro(4,5)decane-6-oxyl, 2,2,6,6-tetramethyl-4-acetoxypiperidine-1-oxyl, and 2,2,6,6-tetramethyl-4-benzoyloxypiperidine-1-oxyl. The amount of the polymerization inhibitor as added is favorably in the range of 0.0001 to 1 weight %, more favorably 0.001 to 0.5 weight %, of the (meth)acrylic acid.

There is no limitation with catalysts as used in the reaction, conventional catalysts as used in this kind of reaction can be used. Examples thereof include at least one member selected from the group consisting of: iron compounds such as iron powder, ferric chloride, iron formate, iron acetate, iron acrylate, and iron methacrylate; chromium compounds such as sodium bichromate, chromium chloride, chromium acetylacetonate, chromium formate, chromium acetate, chromium acrylate, chromium methacrylate, sodium, and chromium dibutyldithiocarbamate; and amines, such as trialkylamines and ion-exchangers having quaternary ammonium groups. However, among them, a basic resin is favorably used as the catalyst. This basic resin means a polymer compound that has a basic functional group and is insoluble in the reaction liquid (for example, a compound having a molecular weight of not less than 1,000). Examples thereof include: polymer compounds having basic functional groups, such as tertial amines, quaternary ammonium salts, cyclic amines (for example, pyridine) and sulfides. Basic anion exchange resins, especially anion exchange resins having amino groups as the basic functional group is favorable. In addition, the above-mentioned iron compounds or chromium compounds may be used together.

The amount of the catalyst used for carrying out the present invention is not especially limited, but, when the catalyst is a heterogeneous catalyst and the reaction is carried out in a batch manner, the catalyst is usually used in the range of 5 to 50 weight %, particularly favorably 10 to 30 weight %, of the raw (meth)acrylic acid. In addition, when the reaction is carried out in a continuous manner and the tank reactor is used in a fluidized bed manner, the catalyst is used in the range of 5 to 90 vol %, favorably 10 to 80 vol %, more favorably 20 to 80 vol %, of the volume of the reaction liquid. In addition, when the tubular reactor is used in a fixed bed manner, the liquid including the raw reaction materials is passed through at a liquid hourly space velocity (LHSV: hr⁻¹) of 0.05 to 15, favorably 0.2 to 8. On the other hand, when the catalyst is a homogeneous catalyst, the catalyst is usually used in the range of 0.05 to 10 weight %, particularly favorably 0.1 to 3 weight %, of the raw (meth)acrylic acid.

Next, the recovery and recycling of the unreacted (meth)acrylic acid, which is characteristic of the production process for a hydroxyalkyl (meth)acrylate according to the present invention, is explained.

The production process for a hydroxyalkyl (meth)acrylate, according to the present invention, comprises the step of carrying out the reaction between the (meth)acrylic acid and the alkylene oxide in order to produce the hydroxyalkyl (meth)acrylate, and further comprises the steps of: recovering the unreacted (meth)acrylic acid by distillation of the resultant reaction liquid; and thereafter recycling the recovered unreacted (meth)acrylic acid as a raw material for the reaction.

As to the technology concerning the separation, recovery and recycling of the unreacted alkylene oxide, a method, which involves separating by stripping and recovering by absorbing solvents, was disclosed in the past (JP-A-330320/1998, JP-A-240853/1999, Japanese Patent Application No. 2000-22692, and Japanese Patent Application No. 2000-32340). On the other hand, the (meth)acrylic acid has a strong affinity for the hydroxyalkyl (meth)acrylate, and a small relative volatility, and further, is easily polymerizable alone or in a mixed solution. Therefore, the technology concerning the separation, recovery and recycling of the unreacted (meth)acrylic acid, was not completed in the past. In the present invention, the separation, recovery, and recycling of the unreacted (meth)acrylic acid, which was difficult to carry out in the past, can be realized by carrying out a specific procedure.

The distillation of the reaction liquid in the present invention is favorably carried out under an operational pressure of 1 to 40 hPa. In the case where the pressure is lower than 1 hPa, the saturation temperature of the vapor including the (meth)acrylic acid lowers at the top of the distillation column, and it is difficult to condense and recover this vapor by passing it through a condenser with cooling water. In addition, in the case where the pressure is higher than 40 hPa, there are disadvantages in that: the temperature rises in the distillation column, especially at the bottom of the distillation column; the polymerization and clogging are caused in the column while the distillation procedure is carried out; and then the procedure is stopped. The (meth)acrylic acid can effectively be separated, recovered, and recycled without causing the polymerization under an operational pressure of 1 to 40 hPa if the distillation is carried out in the presence of the polymerization inhibitors as shown in the aforementioned reaction and oxygen, wherein examples of the polymerization inhibitors include: phenol compounds such as hydroquinone; paraphenylenediamines such as N,N′-di-2-naphthyl-para-phenylenediamine; amine compounds such as phenothiazine; copper dialkyldithiocarbamates such as copper dimethyldithiocarbamate; nitroso compounds, such as N-nitroso-N-phenyl-N-hydroxylamine, or their salts; and N-oxyl compounds, such as 4-hydroxy-2,2,6,6-tetramethylpiperidine-1-oxyl. If, of these polymerization inhibitors, at least one compound selected from the group consisting of the above polymerization inhibitors other than the N-oxyl compounds is used jointly with the N-oxyl compounds, the remarkable effect of inhibiting the polymerization can be obtained.

When the reaction is carried out in a batch manner, the distillation of the reaction liquid may be carried out in a batch or continuous manner in the present invention. When the distillate from the distillation is recovered and recycled as the raw reaction materials, an intermediate tank for the distillate is required in any case of the distillation manner.

When the reaction is carried out in a continuous manner, the distillation of the reaction liquid may also be carried out in a batch or continuous manner in the present invention. However, when the distillation is carried out in a batch manner, a tank for the distillation supply and a tank for the distillate are required. Therefore, the continuous manner is favorable.

In the present invention, the distillation of the reaction liquid may favorably be carried out with a rectifying column, especially with a plate column and/or a packed column. The amount of the distillate is comparatively decreased because these columns are used. In addition, the concentration of the a hydroxyalkyl (meth)acrylate is comparatively lowered in the distillate. Therefore, the reaction yield is decreased a little even if this distillate is recycled in the reaction. In addition, it is favorable that the volume of the reaction apparatus is comparatively diminished.

Even a flash distillation apparatus can separate the unreacted (meth)acrylic acid from the reaction liquid if the amount of the distillate is increased. However, the concentration of the (meth)acrylic acid is lowered in the distillate, and on the other hand, the concentration of the hydroxyalkyl (meth)acrylate is raised. Then, when a comparatively large quantity of the distillate having a high concentration of the hydroxyalkyl (meth)acrylate is recycled in the reaction, the concentration of the hydroxyalkyl (meth)acrylate is raised in the reaction apparatus, and the reaction rate is decreased because the concentration of the raw material is lowered. Therefore, the formation of the diester or dialkylene glycol mono(meth)acrylate is increased, and the reaction yield is decreased. Furthermore, the reaction time is prolonged because the reaction rate is decreased. In addition, it results in comparatively enlarging the volume of the reaction apparatus.

In the production process according to the present invention, the concentration of the (meth)acrylic acid is favorably in the range of 0.1 to 20 weight % in the reaction liquid. In the case where the concentration of the (meth)acrylic acid is higher than 20 weight % in the reaction liquid, the required number of the plate or the necessary packing height for the rectifying column which is a favorable example of the distillation column to separate the (meth)acrylic acid from the reaction liquid is required to increase. However, there are disadvantages in that the polymerization may be caused in the column because the pressure of the column bottom is raised in this case, and therefore, the temperature is also raised. In addition, in the case where the concentration of the (meth)acrylic acid is lower than 0.1 weight % in the reaction liquid, there are disadvantages in that the by-products such as the diester and dialkylene glycol mono(meth)acrylate are rapidly formed in the reaction.

The production process for a hydroxyalkyl (meth)acrylate, according to the present invention, further comprises the steps of: recovering the unreacted (meth)acrylic acid by distillation of the reaction liquid resultant from the reaction between the (meth)acrylic acid and the alkylene oxide; and thereafter recycling the recovered unreacted (meth)acrylic acid as a raw material for the reaction. In these steps, it is also possible that the unreacted alkylene oxide which cannot entirely be separated in the alkylene oxide separation step is recovered and recycled together with the unreacted (meth)acrylic acid. That is to say, it is possible that the unreacted alkylene oxide, which is included in the liquid supplied to the distillation column, is separated into the column top in the above distillation procedure and thereafter passed through a condenser, and that a portion thereof thereafter dissolves into the distillate so as to be recovered.

In the production process for a hydroxyalkyl (meth)acrylate according to the present invention, the distillate as obtained by the distillation of the reaction liquid (including the unreacted (meth)acrylic acid) is recycled in the following different modes when the reaction is carried out in a batch manner or when the reaction and the distillation are carried out in a continuous manner.

When the reaction is carried out in a batch manner, the distillate is once stored in the intermediate tank for the distillate. When the distillation of the reaction liquid is carried out with a rectifying column, such as a packed column, a plate column, or a perforated-plate column, the liquid in the intermediate tank is supplied to the rectifying column as a reflux while the distillation is carried out. When the subsequent batch reaction is carried out, the residual distillate is supplied to a reactor and recycled as the raw reaction material. The method for supplying to the reactor may involve adding it, in a lump before the reaction, or continuously or intermittently while the reaction is carried out. In addition, when the unreacted alkylene oxide is recovered and recycled by absorption, this distillate may be supplied to the reactor after the distillate is used as an absorbing liquid.

When the reaction is carried out in a continuous manner, the distillate may be stored once in the intermediate tank for the distillate or directly introduced to a feeding pump continuously. When the distillation of the reaction liquid is carried out with a rectifying column, such as a packed column, a plate column, or a perforated-plate column, a portion of the distillate is supplied to the rectifying column as a reflux, and a portion of the residual distillate supplied to a reactor and recycled as the raw reaction material. The method for supplying to the reactor may involve adding after the mixing of the raw (meth)acrylic acid and the raw alkylene oxide or adding from separate addition lines. In addition, when the unreacted alkylene oxide is recovered and recycled by absorption, this distillate may be supplied to the reactor after the distillate is used as an absorbing liquid.

In the production process according to the present invention, the resultant crude hydroxyalkyl ester may further be purified when the occasion demands. The method for purifying is not especially limited, but examples thereof include purification by distillation, more particularly, by distillation with conventional flash distillation apparatuses, distillation columns, or rectifying columns, such as packed columns, bubble-cap columns, or perforated-plate columns. However, the distillation is not especially limited to these columns. In addition, other means of distillation can be carried out in addition to the purification by distillation. (Effects and Advantages of the Invention):

The present invention can provide a novel production process for a hydroxyalkyl (meth)acrylate in which, when the hydroxyalkyl (meth)acrylate is produced by carrying out a reaction between (meth)acrylic acid and an alkylene oxide, the unreacted (meth)acrylic acid can be effectively recovered and thereafter recycled as a raw reaction material.

### DETAILED DESCRIPTION OF THE PREFERRED EMBODIMENTS

Hereinafter, the present invention is more specifically illustrated. However the present invention is not limited to these examples.

### EXAMPLE 1

First of all, 480 ml of anion exchange resin (DIAION PA316, made by Mitsubishi Chemical Co., Ltd.) as a catalyst was added to an autoclave equipped with stirring blades, and ethylene oxide, and acrylic acid including hydroquinone monomethyl ether in an amount of 1.0 weight % were continuously added thereto with feeding speeds of 229 g/h and 260 g/h respectively. Then, the reaction for producing hydroxyethyl acrylate was carried out under a condition that the reaction temperature was 70° C. and the reaction time was 4.1 hr. The concentration of oxygen was maintained at 3 mol % in the autoclave in order to inhibit polymerization. The pressure was about 4,200 Pa while the reaction was carried out.

This reaction liquid was continuously supplied to a packed column having a packing height of 32 cm from its column top, and the unreacted ethylene oxide was stripped and separated by supplying nitrogen from the bottom of the column with a feeding speed of 72 g/h in order to inhibit polymerization, wherein the concentration of oxygen was adjusted to 3 mol % in the nitrogen.

Furthermore, the liquid from the bottom of a stripping column was continuously supplied to the third plate of a plate column having seven plates, and the rectification was carried out under a condition that: the operational pressure, the temperature of the column top, the reflux ratio, and the distillate ratio were 7.0 hPa, 74° C., 1.5, and 25%, respectively. When the reflux was carried out, hydroquinone monomethyl ether was added so that the concentration of the hydroquinone monomethyl ether would be adjusted to 0.1 weight % in the reflux. Air was supplied from the bottom of the column with a feeding speed of 0.6 g/h in order to inhibit polymerization. This distillate was continuously supplied to the autoclave again with a feeding speed of 140 g/h.

These series of procedures were continuously carried out for nine hours. After a stationary state was reached, the reaction liquid from the outlet of the autoclave was analyzed. As a result, the conversion of the acrylic acid, the amount of the unreacted acrylic acid, and the amount of the unreacted ethylene oxide were 90.0%, 4.6 weight %, and 11.2 weight %, respectively. Then, the liquid from the bottom of the plate column was analyzed in the same way. As a result, the concentration of the acrylic acid was 0.055 weight %. This concentration corresponded to the recovery efficiency of the unreacted acrylic acid of 99.0%. When the distillate was analyzed at the top of the plate column, the concentration of the acrylic acid was 20.7 weight % and the concentration of the ethylene oxide was 610 ppm. This corresponded to the recovery efficiency of the unreacted ethylene oxide of 6%. Incidentally, the temperature of the bottom of the plate column was then 105° C. However, the polymerization was not caused in the plate column and the operation could be carried out stably.

### EXAMPLE 2

The procedure was carried out in the same way as of Example 1 except that the operational pressure of the plate column was adjusted to 40 hPa. After the operation was continued for 9 hours, the liquid from the bottom of the plate column was analyzed. As a result, the concentration of the acrylic acid was 0.055 weight %. This corresponded to the recovery efficiency of the unreacted acrylic acid of 99.0%. Incidentally, the temperature of the bottom of the plate column was then 125° C. However, the polymerization was not caused in the plate column and the operation could be carried out stably.

### EXAMPLE 3

The procedure was carried out in the same way as of Example 1 except that the operational pressure of the plate column was adjusted to 1 hPa. After the operation was continued for 9 hours, the liquid from the bottom of the plate column was analyzed. As a result, the concentration of the acrylic acid was 0.055 weight %. This corresponded to the recovery efficiency of the unreacted acrylic acid of 99.0%. Incidentally, the temperature of the top of the plate column was then 45° C. and the temperature of the inlet of cooling water was 30° C. However, the amount of the condensed liquid, which was collected in a trap (ice and salt) as arranged at downstream of the condenser and kept at about −20° C., was 6 g. This 15 corresponded to the uncondensed loss of 0.5% in the distillation.

### Comparative Example 1

The procedure was carried out in the same way as of Example 1 except that the operational pressure of the plate column was adjusted to 50 hPa. After the operation was continued for 9 hours, the liquid from the bottom of the plate column was analyzed. As a result, the concentration of the acrylic acid was 0.071 weight %. This corresponded to the recovery efficiency of the unreacted acrylic acid of 98.7%. Incidentally, the temperature of the bottom of the plate column was then 138° C. The column was disassembled after the operation of the plate column was ceased. Then, about 15 g of a polymerized product was formed in all on the plates and at the bottom of the column.

### Comparative Example 2

The procedure was carried out in the same way as of Example 1 except that the operational pressure of the plate column was adjusted to 0.5 hPa. After the operation was continued for 9 hours, the liquid from the bottom of the plate column was analyzed. As a result, the concentration of the acrylic acid was 0.055 weight %. This corresponded to the recovery efficiency of the unreacted acrylic acid of 99.0%. Incidentally, the temperature of the top of the plate column was then 35° C. and the temperature of the inlet of cooling water was 30° C. However, the amount of the condensed liquid, which was collected in a trap (ice and salt) as arranged at downstream of the condenser and kept at about −20° C., was 15 g. This corresponded to the uncondensed loss of 12.1% in the distillation.

Various details of the invention may be changed without departing from its spirit not its scope. Furthermore, the foregoing description of the preferred embodiments according to the present invention is provided for the purpose of illustration only, and not for the purpose of limiting the invention as defined by the appended claims and their equivalents.

What is claimed is:

1. A production process for a hydroxyalkyl (meth)acrylate, which comprises the step of carrying out a reaction between (meth)acrylic acid and an alkylene oxide in order to produce the hydroxyalkyl (meth)acrylate,
with the production process further comprising the steps of: recovering the unreacted (meth)acrylic acid by distillation of the resultant reaction liquid; and thereafter recycling the recovered unreacted (meth)acrylic acid as a raw material for the reaction.

2. A production process according to claim 1, which further comprises the steps of: recovering the unreacted alkylene oxide together with the unreacted (meth)acrylic acid; and thereafter recycling them.

3. A production process according to claim 1, which further comprises the steps of: separating the unreacted alkylene oxide from the reaction liquid in the first place; and thereafter recovering the unreacted (meth)acrylic acid by the distillation.

4. A production process according to claim 1, wherein the distillation is carried out under an operational pressure of 1 to 40 hPa.

5. A production process according to claim 1, wherein the distillation is carried out with a plate column and/or a packed column.

6. A production process according to claim 1, wherein the distillation is carried out in the presence of polymerization inhibitors involving the joint use of at least one compound with an N-oxyl compound wherein the at least one compound is selected from the group consisting of phenol compounds, paraphenylenediamines, amine compounds, copper dialkydithiocarbamates and nitroso compounds.

7. A production process according to claim 1, wherein the concentration of the (meth)acrylic acid in the reaction liquid is in the range of 0.1 to 20 weight %.
